(19) United States
(12) Patent Application Publication
(10) Pub. No.: US 2010/0047360 A1
(43) Pub. Date: Feb. 25, 2010

(54) Use Of Polyamines In The Treatment Of Psoriasis

(75) Inventors: Jo Klaveness, Oslo (NO); Geir Havard Kvalheim, Oslo (NO)

Correspondence Address: Ballard Spahr LLP, SUITE 1000, 999 PEACHTREE STREET, ATLANTA, GA, 30309-3915, US

(73) Assignee: Bioforskining Pharma AS, Oslo (NO)

(21) Appl. No.: 12/225,060

(22) PCT Filed: Mar. 14, 2007
(86) PCT No.: PCT/GB2007/000894

§ 371 Date: Jun. 2, 2009

(30) Foreign Application Priority Data
Mar. 14, 2006 (GB) 0605107.2

(51) Int. Cl. C07C 211/01; A61K 31/132; A61K 31/16; C07C 229/00; A61K 31/205; A61K 38/43; A61K 35/12; A61K 36/00; A61K 38/39; A61P 17/06

(52) U.S. Cl. 424/520; 564/512; 514/674; 564/511; 514/626; 554/104; 514/554; 514/171; 424/94.1; 424/725; 514/12

(57) ABSTRACT

The present invention provides unbranched aliphatic polyamines for use in therapy, particularly for use in the treatment of psoriasis and compositions containing polyamines.

This invention relates to the use of spermine and other polyamines for treatment of psoriasis.

Psoriasis is an inflammatory skin disease which affects about 2% of the adult population. It generally causes scaly lesions which may be sore, itchy and/or irritated. Plaque psoriasis is the most common form of the disease and typically affects the skin of the scalp, lower back and extensor aspects of the limbs. Other variants include, sebopsoriasis, guttate psoriasis, pustular forms, inverse psoriasis and psoriatic arthritis.

The pathogenesis of the condition is not fully understood and no cure is currently known. There are several therapies available today, including systemic therapy, topical therapy and UV light. The most popular therapies include drugs like methotrexate, retinoids, cyclosporine, glucocorticoids and analogues of 1,25-dihydroxyvitamin D₃.

Drugs for psoriasis can be administered systemically or locally on the affected skin Topical drugs for psoriasis treatment include glucocorticoids, vitamin D₃ analogues, retinoids, coal tar and anthralin. Topical application is often useful when smaller areas of the skin is affected, while systemic or normally oral administration of antipsoriasis drugs are preferred when psoriasis affects larger areas. UV therapy is often used when larger areas are affected and this radiation therapy is often performed in presence of a sensitizer like psoralen. Systemic antipsoriasis drugs include cyclosporine, methotrexate and fumaric acid esters.

Several new drugs for treatment of psoriasis have been suggested or are in development. These include new compounds with affinity for the nuclear vitamin D receptor, new compounds that inhibit the expression of psoriasis related genes, trimetrexate and other methotrexate analogues, immunosuppressive agents, tacrolimus, ascomucines, metabolic inhibitors of retinoic acid; for example cytochrome P-450 inhibitors, inhibitors of inositol-5-monophosphate dehydrogenase, leukotriene B4 antagonists, antisense oligonucleotides, protein tyrosine kinase inhibitors, nuclear receptor ligands, inhibitors of cytokine, inhibitors of growth factors like EGFR inhibitors, and blockers of T-cell migration and adhesion. Other drugs in development for treatment of psoriasis include biological immune response modifiers.

However, all current therapies have drawbacks related to lack of efficacy or severe side effects both of which lead to poor patient compliance. There thus exists a need for new alternatives therapies.

It is well known in the art that levels of polyamines, i.e. polyazaalkanes, such as spermine (1,5,10,14-tetraazatetradecane) are elevated in psoriasis patients, e.g. in the skin (J. Invest. Dermatology, 80, 181-184 (1983)) and blood (J. Invest. Dermatology, 71, 177-181 (1978); Life Science, 19, 257-264 (1976)). It is also well documented that, upon effective treatment of psoriasis, the elevated polyamine levels are seen to decrease (J. Am. Acad. Dermatology, 8, 95-102 (1983); British J. Dermatology, 105, 267-272 (1981); Eur. J. Clin. Invest., 8, 215-218 (1978)). This correlation between psoriasis and polyamine levels has led to investigations into compounds able to inhibit the synthesis of polyamines as lowering polyamine levels is believed to contribute to the treatment of psoriasis (Archives of Dermatology, 115, 945-949 (1979); (J. Invest. Dermatology, 80, 181-184 (1983)).

In contrast to the teaching that lowering polyamine levels is effective in the treatment of psoriasis, it has been unexpectedly found that the symptoms of psoriasis in patients can be relieved using a formulation containing polyamines. The present invention therefore relates to the use of polyamines in the treatment of psoriasis.

In a first embodiment the invention therefore provides polyamine compositions for use in therapy. Preferably the polyamine is an unbranched aliphatic polyamine. A further aspect of the invention is the use of polyamine compositions in the manufacture of a medicament for the treatment of psoriasis. A further aspect of the invention provides a method of combating psoriasis comprising administering to a subject an effective amount of a polyamine composition according to the invention.

Viewed from a further aspect the invention provides the use of an unbranched aliphatic polyamine for the manufacture of a topical skin treatment composition for use in the topical treatment of psoriasis.

Viewed from a further aspect the invention provides a method of treatment of a subject to combat psoriasis, which method comprises topically applying to the skin of said subject an effective amount of an unbranched aliphatic polyamine.

Viewed from a further aspect, the invention provides polyamine compositions as described herein, preferably emulsions of polyamines, preferably unbranched aliphatic polyamines, and/or salts thereof. The emulsion of the polyamine or polyamine salt may be an oil-in-water or a water-in-oil emulsion.

The invention also provides pharmaceutical compositions comprising polyamines, preferably unbranched aliphatic polyamines and/or salts thereof in combination with one or more skin penetration enhancing agents. Suitable skin penetration enhancers are propylene glycol laurate, propylene glycol monolaurate, propylene glycol monocaprylate, sodium lauryl sulphate, phospholipids, alcohols (such as ethanol, ispropanol, n-octanol and decanol), N-methyl-2-pyrrolidone, Tween 80 and other surfactants and solvent such as DMSO.

In the above-mentioned emulsions and pharmaceutical compositions the polyamine (or salt thereof) is preferably present in amounts of less than 10% wt, preferably 0.0005 to 5% wt, more preferably 0.001 to 1% wt, especially 0.005 to 0.5% wt, particularly 0.01 to 0.08% wt, more particularly 0.02 to 0.06% wt, especially 0.03 to 0.05% wt, e.g. 0.04% wt (i.e. 400 ppm). A total polyamine content of 0.005-0.05% wt is particularly preferred.

The emulsions and pharmaceutical compositions herein described are particularly effective against psoriasis.

The polyamine used according to the invention will generally be an isolated pure substance, formulated in a sterile composition with appropriate cosmetic or pharmaceutical carriers or excipients.

The subject treated according to the invention may be any mammal, but humans are intended as the normal subjects.

In an especially preferred embodiment, the method of the invention is a method of treatment of a subject to combat psoriasis, which method comprises topically applying to the skin of said subject, e.g. a subject having visible psoriatic lesions, an effective amount of an unbranched aliphatic polyamine.

Viewed from a yet further aspect the invention provides a topical skin treatment composition comprising an unbranched aliphatic polyamine and at least one physiologically tolerable carrier or excipient, together with instructions for the topical application thereof to combat psoriasis. Such instructions may typically be provided on the external packaging, as an insert within the external packaging or on the composition container itself.

Preferably the composition of, or for use in, the invention comprises at least one physiologically tolerable carrier or excipient, a first unbranched aliphatic polyamine and a further active agent selected from the group consisting of: polyazaalkanes other than said first unbranched aliphatic polyamine, dimethyl sulphoxide, keratolytic agents, unsaturated fatty acids (e.g. omega-3, omega-6 and omega-9 unsaturated fatty acids, especially omega-3 acids, for example EPA, DHA and ALA) and derivatives (particularly esters) thereof, HMG-CoA reductase inhibitors, piperic acid, 8-hexadecene-1,16-dicarboxylic acid, natural triterpenes, Coenzyme Q10 (ubiquinone), vitamin B3, aloe, acetylglucosamine esters, ACE inhibitors, angiotensin receptor antagonists, eugenyl glycosides, Mallotus japonicus extract, hydroxyacids (e.g. alpha hydroxy acids such as glycolic acid), beta-(1,3) glucans, frog extract, extract of unpolished rice, urea, pine seed oil, marine collagens, plant cell extracts, ursolate and eugenol derivatives, ceramides, cholesterol, glutathione, carnitine, oxygen scavangers, phytosphingosine, calcium channel inhibitors, sucrose linolenate, caffeine, catalase, Rosa mosqueta oil, glycine, Shea butter, perfluoro polyethers, cystein derivatives, and acetylated hyaluronic acid and alpha-amino acids, and salts of any of these.

Particularly preferred active ingredients besides the polyazaalkanes include Coenzyme Q10, Vitamin B3, alpha-hydroxy acids, unsaturated fatty acids (e.g. omega-3, omega-6 and omega-9 unsaturated fatty acids, especially omega-3 acids, for example EPA, DHA and ALA) and derivatives (particularly esters) thereof, catalase, and Rosa mosqueta oil.

In particular the composition contains two or more unbranched aliphatic polyamines; or an unbranched aliphatic polyamine and catalase; or an unbranched aliphatic polyamine and vitamin B3; or an unbranched aliphatic polyamine and Rosa mosqueta oil; or an unbranched aliphatic polyamine and coenzyme Q10; or an unbranched aliphatic polyamine and an unsaturated fatty acid (e.g. an omega-3, omega-6 and omega-9 unsaturated fatty acid, especially an omega-3, for example EPA, DHA and ALA) or a derivative (particularly an ester) thereof; or an unbranched aliphatic polyamine and an alpha hydroxy acid.

Viewed from a further aspect the invention provides salts of aliphatic polyamines (e.g. linear α, ω-diaminoalkanes or -mono, di or polyazaalkanes, typically having C₂₋₅ alkylene chains between the nitrogens) with fatty acids (typically with saturated, mono-unsaturated or polyunsaturated linear C₁₆₋₂₄ chains attached to a carboxyl group) and compositions thereof with at least one physiologically tolerable carrier or excipient. Examples thus include salts of spermine with EPA or DHA. Such salts can readily be prepared by reaction of the polyamine and the fatty acid in a solvent or solvent mixture in which they are at least partially soluble, e.g. an organic solvent.

The polyamines used according to the present invention are preferably amine group terminated linear structures. Desirably they are unbranched aliphatic compounds which occur naturally. The polyamines preferably have (CH₂)ₙ groups linking the nitrogens where n is 2 to 6, especially 3 or 4, and particularly ones comprising 2 to 6 nitrogens, particularly 2, 3 or 4 nitrogens. These polyamines are available from natural sources, e.g. mammalian semen or fermentation products (for example from soy or anchovies), or may be manufactured by conventional techniques, e.g. solid state polypeptide production followed by amidation and reduction. It is particularly preferred to use naturally occurring polyamines, e.g. putrescine (H₂N(CH₂)₄NH₂), cadaverine (H₂N(CH₂)₅ NH₂), spermidine (H₂N(CH₂)₃NH(CH₂)₄NH₂), and spermine (H₂N(CH₂)₃NH(CH₂)₄NH(CH₂)₃NH₂), more particularly putrescine, spermidine or spermine, and especially spermine. The use of a combination of two such polyamines, e.g. in a mole ratio of 1:99 to 99:1 especially 10:90 to 90:10, is especially preferred (e.g. spermine and spermidine) as is the use of a combination of three or more such polyamines, for example with each present at 1 to 100% mole relative to the most abundant, especially 10 to 100% mole, particularly 30 to 100% mole.

The use of dibutylenetriamine, tributyltetramine, 1,6,10,15-tetraazapentadecane, 1,5,9,13-tetraazatridecane and 6-aminobutyl-1,6,11-triazaundecane may also be considered.

In the polyamines used according to the invention the average carbon chain length, i.e. the carbon chain between heteroatoms, may be as low as 1 or 2; however, where this average is below 3.0 the polyamine is preferably a minor component of the composition, e.g. no more than 5% wt, preferably no more than 1% wt.

In general, in the polyamines of the invention the average carbon chain length is preferably at least 2.5, more preferably at least 3.0, especially at least 3.25, e.g. 3.25 to 6.0.

Desirably the polyamines used according to the invention have molecular weights in the range 88 to 202 Da.

The polyamine used in accordance with the invention may conveniently be in salt form with a physiologically tolerable counterion, e.g. an organic acid, particularly preferably an alpha-hydroxyacid or fatty acid.

In the compositions of, or used according to, the invention the total polyamine content is usually less than 10% wt, preferably 0.0005 to 5% wt, more preferably 0.001 to 1% wt, especially 0.005 to 0.5% wt, particularly 0.01 to 0.08% wt, more particularly 0.02 to 0.06% wt, especially 0.03 to 0.05% wt, e.g. 0.04% wt (i.e. 400 ppm). A total polyamine content of 0.005-0.05% wt is particularly preferred. The final concentration of the polyamine in the formulation is dependent on the nature of the psoriasis disease, choice of polyamine compound and composition of the formulation.

The compositions of, or used according to, the invention preferably do not contain multivalent metal (e.g. transition metal) ions in otherwise labile form at concentrations of above 10% mole relative to the polyamine, especially 1% mole.

For topical application, the compositions of, or used according to, the invention may be in any form suitable for topical application, e.g. creams, gels, solutions, emulsions, dispersions, suspensions etc. and may if desired include a carrier substrate, e.g. a woven or non-woven web. Emulsions (either oil-in-water or water-in-oil) are especially preferred. The compositions may contain conventional topical composition components, such as for example, solvents, oils (e.g. plant oils), aromas, colorants, pH modifiers, viscosity modifiers, binders, diluents, emollients, antioxidants, skin irritants, thickeners, vitamins, preservatives, stabilizers, humidifiers, skin penetration enhancers, vesicle wall formers, etc. Examples of suitable formulations include body milks, body lotions, hand creams and oils. The compositions used according to the invention are particularly preferably creams, emulsions, gels, vesicle dispersions, or vesicle forming compositions. In terms of vesicles, liposomes are of particular interest as they can facilitate skin penetration of the polyamine. Liposome formulations may be prepared conventionally, e.g. using commercially available precursors. Equally, the inclusion of keratolytics and skin penetration enhancers, e.g. DMSO, is of particular interest, as is the inclusion of vitamins such as vitamin A, vitamin C, vitamin B₆ and vitamin E and derivatives thereof.

The inclusion of a skin penetration enhancer in the polyamine compositions of the present invention is especially preferred. Suitable skin penetration enhancing agents are e.g. propylene glycol laurate, propylene glycol monolaurate, propylene glycol monocaprylate, sodium lauryl sulphate, phospholipids, alcohols (such as ethanol, ispropanol, n-octanol and decanol), N-methyl-2-pyrrolidone, Tween 80 and other surfactants and solvent such as DMSO.

Inclusion of skin penetration enhancing agents is particularly preferred when the composition is an emulsion or a homogeneous water-based formulation. For homogeneous water-based formulations, skin penetration enhancing agents such as alcohols (e.g. ethanol, isopropanol) or DMSO are preferred.

The components of the compositions of, or used according to, the invention will typically be present in conventional concentrations for skin treatment compositions. Active components, i.e. those having a skin protective effect beyond simple moisturization or oiling, will generally be present at concentrations of 0.001 to 20% wt, especially 0.01 to 10% wt, particularly 0.05 to 5% wt.

As the polyamines may be electrically charged, e.g. by the inclusion of quaternary amine functions or by protonation of amine nitrogens, the compositions may deliver the polyamine transdermally under the action of an electric field, i.e. by iontophoresis. The compositions may thus conveniently be presented in gel form within patches provided with electrodes and a battery. This format is of particular interest when the skin treatment desired is localized, e.g. in the treatment of localized lesions.

In general, the compositions should be applied to the skin either prophylactically, i.e. to inhibit development of a lesion, or to the affected skin of a subject in which the psoriatic lesion is already present.

The polyamines will typically be administered at a dosage of about 0.01 to 50 g/m², preferably 0.1 to 10 g/m², especially 1 to 5 g/m². Any other active ingredients will typically be used at from 10% to 200%, preferably 50 to 110%, more preferably 80 to 105% of their normal dosages.

The compositions of, or used according to, the invention may be produced by standard pharmaceutical composition production techniques, e.g. simple admixture optionally followed by sterilization. The compositions are desirably packaged in single dose units or in units suitable for up to 100 applications, e.g. 2 to 10 applications. The use of sachets, spray dispensers, pump dispensers, and wipes is especially preferred.

A further preferred aspect of the present invention relates to compositions comprising spermine or other polyamines for systemic administration for treatment of psoriasis. The most preferred systemic compositions comprising polyamines for treatment of psoriasis include oral formulations like tablets, capsules and solutions and injection solutions/suspensions. The amount of polyamine in one tablet or one capsule can vary over a large range; preferably from 1 mg to 1 g; most preferably from 5 mg to 500 mg. An oral solution or solution for injection contains preferably 1-200 mg polyamine per ml; most preferably 2-100 mg polyamine per ml. In solutions of polyamines the polyamines are preferably in the form of a salt with high solubility in water.

One preferred aspect of the present invention relates to formulations for systemic use comprising a combination of a polyamine such as spermine with other therapeutically active drugs with activity against psoriasis. Typical such formulations could for example be polyamine together with methoxalen, polyamines together with acitrectin, polyamines together with methotrexate and polyamines together with ciclosporamin. The polyamines can in these formulations be in the form of free base or in the form of a salt with a physiologically acceptable inorganic or organic acid. One preferred such formulation comprises of polyamine salt with acids that are active in treatment of psoriasis; for example spermine methotrexate salt, spermine fumarate salt and spermidine fumarate salt.

Spermine, spermidine, and other polyamines can be used in the form of free base or in the form of a salt with a physiologically acceptable inorganic or organic acid for example in the form of the HCl or HBr salt.

Another preferred aspect of the present invention relates to polyamine salts with antipsoriatic active acids like for example retinoic acid.

One preferred aspect of the present invention is compositions comprising a polyamine together with a second pharmacological active substance for treatment of psoriasis.

One preferred such composition combines for example spermine and glucocorticoids. The most preferred glucocorticoids to be combined with polyamines like spermine include hydrocortisone, desonide, dexamethasone, hydrocortisone valerate, triamcinolone acetonide, betamethasone valerate, flurandrenolide, mometasone furoate, flucoinonide, halcinonide, amcinonide, desoximetasone, diflorosone diacetate, halobetasol propionate, betamethasone dipropionate and clobetasol dipropionate.

The concentration of glucocorticoids in topical compositions comprising polyamines varies from 0.05% to 3% hydrocortisone and other corticosteroids are typically present up to 3% while fluocinonide and other high potent glucorticosteroids with low potency are normally in the range of approximately 0.05-0.1%.

Another preferred such composition combines for example spermine with vitamin D₃ analogues like 1,25-dihydroxyvitamin D₃. A typical concentration of a vitamin D₃ analogue in a topical formulation according to the present invention is 0.05% (w/w).

Another preferred such composition combines for example spermine with retinoids like tazarotene. A typical concentration of such compounds in topical formulation, according to the present invention is 0.1%.

Another preferred such composition combines for example spermine with dithranol. A typical concentration of dithranol in topical formulations, according to the present invention, is 1%.

Another preferred such composition combines for example spermine with calcitriol. A typical concentration of calcitriol in topical formulation, according to the present invention, is 3 mg/ml composition.

The compositions of the invention may be used (whether topically or systemically) in the treatment of any form of psoriasis (e.g. plaque psoriasis, sebopsoriasis, guttate psoriasis, pustular forms, inverse psoriasis, psoriatic arthritis) and at any stage of the condition. Preferably, the composition according to the invention is used to treat plaque psoriasis.

The invention will now be further described with reference to the following non-limiting examples.

### EXAMPLE 1

Topical cream

| Ingredient | Parts by weight |
|---|---|
| Water | 61-66 |
| Propylene Glycol Dicaprylate/Dicaprate | 6-8 |
| Ethylhexyl Stearate | 3-4 |
| Prunus Armeniaca | 0.5-1.5 |
| Simmondsia Chinensis | 0.4-0.6 |
| C12-20 Acid PEG-8 Ester | 8-12 |
| Olus | 3-4 |
| Propylene glycol | 2.5-3.5 |
| Glyceryl stearate | 1.5-2.5 |
| Potassium cetyl phosphate | 0.8-1.2 |
| Glycerin | 0.4-0.6 |
| Sodium PCA | 0.1-0.2 |
| Dimethicone | 1.5-2.5 |
| Spermine | 0.03 |
| Ascorbyl Palmitate | 0.005-0.015 |
| Ubiquinone | 0.08-0.12 |
| PEG-7 Glyceryl Cocoate | 0.05-0.1 |
| Alcohol denat. | 0.05-0.1 |
| Tocopheryl Acetate | 0.05-0.1 |
| Panthenol | 0.05-0.1 |
| Retinyl Palmitate | 0.05-0.1 |
| Helianthus Annuus | 0.05-0.1 |
| Tocopherol | 0.05-0.1 |
| Lactic Acid | 0.5-1.5 |
| Sodium Gluconate | 0.05-0.15 |
| Phenoxyethanol | 0.4-0.6 |
| Sodium Benzoate | 0.2-0.3 |

The components listed above are mixed and emulsified.

### EXAMPLE 2

Topical cream

| Ingredients | Parts by weight |
|---|---|
| Water | 80-85 |
| Alcohol Denat. | 5-10 |
| Propylene Glycol | 2-4 |
| Sorbitol | 1-3 |
| Polyquaternium-10 | 1-3 |
| Dicaprylyl Carbonate | 0.5-1.5 |
| Sodium Hyaluronate | 0.5-1.0 |
| Tocopherol | 0.05-0.1 |
| Tocopheryl Acetate | 0.05-0.1 |
| Spermine | 0.02-0.04 |
| Ubiquinone | 0.008-0.012 |
| Retinyl Palmitate | 0.05-0.1 |
| PEG/PPG-14/4 Dimethicone | 0.3-0.7 |
| Sodium Gluconate | 0.5-1.5 |
| Menthyl Lactate | 0.05-0.15 |
| Phenoxyethanol | 0.3-0.7 |
| Lactic Acid | 0.5-1.5 |
| Propylene Glycol | 0.008-0.012 |
| Dicaprylate/Dicaprate | |
| Prunus Armeniaca | 0.008-0.012 |
| Panthenol | 0.05-0.1 |
| Helianthus Annuus | 0.05-0.1 |
| PEG-7 Glyceryl Cocoate | 0.05-0.1 |

The components are mixed and emulsified.

Further creams are prepared analogously using the weight content mid-points for these ingredients and further including in parts by weight: (A) 0.03 spermidine; (B) 0.07 vitamin B3; (C) 0.07 catalase; (D) 0.07 Rosa mosqueta oil; (E) 0.03 spermidine, 0.07 vitamin B3; 0.07 catalase and 0.07 Rosa mosqueta oil.

The six compositions of this example may be applied liberally to the skin area to be treated.

### EXAMPLE 3

### Clinical Study—Efficacy

A female patient (age 24) with psoriasis spots all over the body had used various treatments over the last eight years including cream comprising calcipotriol, UVB light treatment and specific travel arrangements for psoriasis patients. The therapeutic effect from use of the calcipotriol cream was low, and, although light treatment and travel arrangements had a relatively good effect, their effects were not permanent. The patient started to use a cream according to Example 1 two to four times daily. Over some weeks she observed a good therapeutic effect and after two to three months treatment the psoriasis spots had almost gone. The patient stopped using the spermine cream after about six months. Some time later some psoriasis spots returned.

### EXAMPLE 4

### Clinical Study—Efficacy

A male patient (age 42) with psoriasis (knees, elbows and scalp) started to use a cream according to Example 1 daily. The patient, who had suffered from psoriasis for 30 years, was free from all psoriasis spots after 3 weeks of treatment.

### EXAMPLE 5

### Clinical Study—Efficacy

A female patient (age 48) with psoriasis (legs, arms, back, face, scalp, and around nails) had previously used several antipsoriasis drugs (steroids, tar, methotrexate) and light treatment. The only effective treatment for this patient was methotrexate. The patient, who had suffered from psoriasis for 25 years, started to use a cream according to Example 1 daily for 4 months. After short timer treatment, the psoriasis spots were substantially reduced, the skin became softer and the itching related to the psoriasis disease was remarkably reduced.

### EXAMPLE 6

### Clinical Study—Efficacy.

A female patient (age 74) with psoriasis (scalp, auditory canal, hands, elbows, partly in the face and body (psoriasis grade medium on 15% of the body)). The patient had previously used steroids without any therapeutic effects. The patient, who had suffered from psoriasis for 15 years, started to use a cream according to Example 1 four times daily. After about 45 days all the psoriasis spots had disappeared.

### EXAMPLE 7

### Clinical Study—Safety

About 3000 individuals (male and female, age from 25 to 85) have used the cream from Example 1 daily in the face. Several of these individuals used the cream several times a day. Less than 1% of the individuals observed an initial mild rash that disappeared. Most of these individuals could continue to use the cream. No other side effect was observed in this study.

### EXAMPLE 8

### Synthesis of Spermine Fumarate (1:2)

Spermine (202 mg; 1.0 mmol) was added to fumaric acid (232 mg; 2.0 mmol) in water (8 mL). The mixture was stirred ca. 15 min., then freeze-dried, leaving 420 mg (98%) white crystals, mp 203-205° (dec.).

### EXAMPLE 9

### Synthesis of Spermidine Fumarate (2:3)

Spermidine (145 mg; 1.0 mmol) was added to fumaric acid (174 mg; 1.5 mmol) in water (8 mL). The mixture was stirred ca. 15 min., then freeze-dried, leaving 310 mg (97%) white crystals, mp 185-188° (dec.).

### EXAMPLE 10

### Capsules for Oral Administration

Spermine fumarate (from Example 8) is mixed with lactose and filled into hard gelatine capsules. Each capsule contains 50 mg spermine fumarate.

### EXAMPLE 11

### Emulsion

An emulsion was prepared by combining spermine (0.04% wt) with Unguentum Merck using a mortar and pestle.

### EXAMPLE 12

### Emulsion

An emulsion was prepared by combining spermine (0.08% wt) with Unguentum Merck using a mortar and pestle.

### EXAMPLE 13

### Emulsion

An emulsion was prepared by combining spermine (0.12% wt) with Unguentum Merck using a mortar and pestle.

### EXAMPLE 14

Clinical Testing of Emulsions from Examples 11-13

A female patient with psoriasis (same patient as in Example 3) used the products from Example 11-13).

All products were effective in treatment of psoriasis.

### EXAMPLE 15

### Preparation of Cream Comprising Lidocaine and Spermine

A cream comprising 0.02% spermine and 5% lidocaine was prepared from Xylocaine® 5% (AstraZeneca) (5 g) and spermine (1 mg) using mortar and pestle.

### EXAMPLE 16

### Preparation of Cream Comprising Hydrocortisone and Spermine

A cream comprising 0.04% spermine and 1% hydrocortisone was prepared from hydrocortisone 1% (Galderma®) cream (10 g) and spermine (4 mg) using mortar and pestle.

### EXAMPLE 17

Hydrogel Comprising Spermine Fumarate and isopropanol

A hydrogel was prepared by dissolution of hydroxyethyl cellulose (2 g) in water (50 g) at 50° C. Propyleneglycol (36 g) was added, followed by addition of isopropanol (12 g). Spermine fumarate (20 mg) (from Example 8) was added to the stirred solution.

### EXAMPLE 18

### Hydrogel Comprising Spermine Fumarate and Dimethylsulfoxide

A hydrogel was prepared by dissolution of hydroxyethyl cellulose (2 g) in water (50 g) at 50° C. Propyleneglycol (36 g) was added, followed by addition of dimethylsulfoxide (12 g). Spermine fumarate (10 mg) (from Example 8) was added to the stirred solution.

1-13. (canceled)

14. An unbranched aliphatic polyamine suitable for use in therapy.

15. The unbranched aliphatic polyamine of claim 1, wherein the therapy comprises treatment of psoriasis.

16. A medicament suitable for use in the treatment of psoriasis, the medicament comprising an unbranched aliphatic polyamine.

17. A method of treatment of a subject to combat psoriasis, which method comprises administering to said subject an effective amount of a medicament comprising an unbranched aliphatic polyazamine.

18. The method according to claim 17 wherein said medicament is administered topically.

19. The method according to claim 17 wherein said medicament is administered systemically.

20. A salt of an aliphatic polyamine with a fatty acid.

21. A pharmaceutical composition comprising a salt as claimed in claim 20 together with at least one pharmaceutical carrier or excipient.

22. A topical skin treatment composition comprising an unbranched polyamine and at least one physiologically tolerable carrier or excipient, together with instructions for the topical application thereof to combat psoriasis.

23. The composition of claim 22, further comprising instructions for the topical application thereof to combat psoriasis.

24. An emulsion of a polyamine or a salt thereof.

25. A pharmaceutical formulation comprising a skin penetration enhancing agent and a polyamine or a salt thereof.

26. The unbranched aliphatic polyamine of claim 14, wherein said polyamine is selected from putrescine, spermidine and spermine.

27. The medicament of claim 16, wherein the total content of polyamine is 0.005-0.05% wt.

28. A composition as claimed in claim 20 further containing a further active agent selected from the group consisting of: polyazaalkanes other than said first unbranched aliphatic polyamine, dimethyl sulphoxide, keratolytic agents, unsaturated fatty acids and derivatives thereof, HMG-CoA reductase inhibitors, piperic acid, 8-hexadecene-1,16-dicarboxylic acid, natural triterpenes, Coenzyme Q10 (ubiquinone), vitamin B₃, aloe, acetylglucosamine esters, ACE inhibitors, angiotensin receptor antagonists, eugenyl glycosides, Mallotus japonicus extract, hydroxyacids, frog extract, extract of unpolished rice, urea, pine seed oil, marine collagens, plant cell extracts, ursolate and eugenol derivatives, ceramides, cholesterol, glutathione, carnitine, oxygen scavangers, phytosphingosine, calcium channel inhibitors, sucrose linolenate, caffeine, catalase, Rosa mosqueta oil, glycine, Shea butter, perfluoro polyethers, cystein derivatives, and acetylated hyaluronic acid and alpha-amino acids, and salts of any of these.
